(19) United States
(12) Patent Application Publication
(10) Pub. No.: US 2005/0153284 A1
(43) Pub. Date: Jul. 14, 2005

(54) Single molecule sequencing method

(76) Inventors: Zeno Foldes-Papp, Graz (AU); Johan Holm, Copenhagen (DK)

Correspondence Address: FULBRIGHT & JAWORSKI, LLP, 666 FIFTH AVE, NEW YORK, NY, 10103-3198, US

(21) Appl. No.: 10/311,673

(22) PCT Filed: Jun. 29, 2001
(86) PCT No.: PCT/EP01/07460

(51) Int. Cl. C12Q 1/68; C12M 1/34

(52) U.S. Cl. 435/6; 435/287.2

(57) ABSTRACT

The invention relates to a method for single molecule sequencing of nucleic acids and to a device suitable for carrying out said method.

The invention relates to a method for single molecule sequencing of nucleic acids and to a device suitable for carrying out said method.

In order to sequence the human genome which consists of approx., 3×10⁹ bases or the genome of other organisms and to determine and compare individual sequence variants, sequencing methods must be provided which, firstly, are rapid and, secondly, can be used routinely and at low cost. Although great efforts have been made in order to accelerate common sequencing methods, for example the enzymic chain termination method according to Sanger et al. (Proc. Natl. Acad. Sci. USA 74 (1977) 5463), in particular by means of automation (Adams et al., Automated DNA Sequencing and Analysis (1994), New York, Academic Press), currently a maximum of only 2,000 bases per day can be determined using a sequencer.

In recent years, novel approaches to overcoming the limitations of conventional sequencing methods have been developed, inter alia sequencing by scanning tunneling microscopy (Lindsay and Phillip, Gen. Anal. Tech. Appl. 8 (1991), 8-13), by highly parallel capillary electrophoresis (Huang et al., Anal. Chem. 64 (1992), 2149-2154; Kambara and Takahashi, Nature 361 (1993), 565-566), by oligonucleotide hybridization (Drmanac et al., Genomics 4 (1989), 114-128; Khrapko et al., FEBS Let. 256 (1989), 118-122; Maskos and Southern, Nucleic Acids Res. 20 (1992), 1675-1678 and 1679-1684) and by matrix-assisted laser desorption/ionization mass spectrometry (Hillenkamp et al., Anal, Chem. 63 (1991), 1193A-1203A).

Another approach is single molecule sequencing (Dörre et al., Bioimaging 5 (1997), 139-152) which comprises sequencing nucleic acids by progressive enzymic degradation of fluorescently labeled single-stranded DNA molecules and detecting the sequentially released monomeric molecules in a microstructure channel in which said monomeric molecules are directed electroosrnotically by pumping. This procedure has the advantage that in each case only a single molecule of the target nucleic acid is sufficient for carrying out a sequence determination.

However, the method described in Dörre et al. has a disadvantage in that the sequentially released monomeric molecules can interact with the walls of said microstructures, and this may cause considerable problems during analysis. It was, therefore, the object of the present invention to provide a method for single molecule sequencing of nucleic acids which represents an improvement compared to the prior art and which, in particular, makes it possible to improve detection by avoiding wall effects.

This object is achieved by a method for sequencing nucleic acids, which comprises the following steps:
(a) providing a support particle on which a nucleic acid molecule has been immobilized, with essentially all nucleotide building blocks of at least one base type in at least one strand of said nucleic acid molecule carrying a fluorescent label,
(b) introducing said support particle into a sequencing device comprising a microchannel,
(c) arresting said support particle in said sequencing device,
(d) progressively removing by cleavage individual nucleotide building blocks from the immobilized nucleic acid molecule,
(e) passing the removed nucleotide building blocks through a microchannel by means of a hydrodynamic flow and
(f) determining the base sequence of said nucleic acid molecule based on the sequence of said removed nucleotide building blocks.

The method of the invention is a sequencing method which comprises studying a single nucleic acid molecule immobilized on a support. The size of the support particle used for said method is such as to enable said support particle to move in microchannels and to be arrested at a desired position within a sequencing device The particle size is preferably in the range from 0.5 to 10 μm and particularly preferably from 1 to 3 μm. Examples of suitable materials of support particles are plastics such as polystyrene, glass, quartz, metals or semimetals such as silicon, metal oxides such as silicon dioxide or composite materials which contain several of the abovementioned components. Particular preference is given to using optically transparent support particles, for example made from plastics, or particles having a plastic core and a silicon dioxide shell.

The nucleic acid molecules are immobilized on the support particle preferably via their 5′ ends. The nucleic acid molecules may be bound to the support via covalent or noncovalent interactions. For example, binding of the polynucleotides to the support can be mediated by high-affinity interactions between the partners of a specific binding pair, for example biotin/streptavidin or avidin, hapten/anti-hapten antibody, sugar/lectin, etc. Thus it is possible to couple biotinylated nucleic acid molecules to streptavidin-coated supports. As an alternative, it is possible to bind the nucleic acid molecules to the support by means of adsorption. Thus, nucleic acid molecules modified by incorporation of alkanethiol groups can bind to metallic supports, for example supports made from gold. Still another alternative is covalent immobilization which comprises the possibility of mediating polynucleotide binding via reactive silane groups on a silica surface.

The method of the invention uses support particles to which only one single nucleic acid molecule is bound. Support particles of this kind may be generated by contacting the nucleic acid molecules intended for sequencing with the support particles in a molar ratio of preferably 1:5 to 1:20, for example 1:10, under conditions under which the nucleic acid molecules are immobilized on the support. The resulting support particles are then sorted, for example on the basis of the fluorescent labeling groups present on the nucleic acid molecules, and removed from particles to which no nucleic acid molecule has bound. Said sorting and removal may be carried out, for example, according to the methods described in Holm et al. (Analytical Methods and Instrumentation, Special Issue μTAS 96, 85-87), Eigen and Rigler (Proc. Natl. Acad. Sci. USA 91 (1994), 5740-5747) or Rigler (J. Biotech. 41 (1995), 177-186), which involve detection by means of a confocal microscope.

The support-bound nucleic acid molecules, for example DNA molecules or RNA molecules, may be present in single-stranded form or double-stranded form. In the case of double-stranded molecules it must be ensured that labeled nucleotide building blocks can be removed by cleavage only from one single strand. In the nucleic acid strands to be sequenced, essentially all nucleotide building blocks, for example at least 90%, preferably at least 95%, of all nucleotide building blocks, of at least one base type carry a fluorescent labeling group. Preferably, essentially all nucleotide building blocks of at least two base types, for example two, three or four base types, carry a fluorescent label, each base type advantageously carrying a different fluorescent labeling group. Nucleic acids labeled in this way may be generated by enzymic primer extension on a nucleic acid template, using a suitable polymerase, for example a DNA polymerase such as, for example, a DNA polymerase from Thermococcus gorgonarius or from other thermostable organisms (Hopfner et al., PNAS USA 96 (1999), 3600-3605) or a mutated Taq polymerase (Patel and Loeb, PNAS USA 97 (2000), 5095-510) and using fluorescently labeled nucleotide building blocks. The labeled nucleic acid strands may also be prepared by amplification reactions, for example PCR. Thus, an asymmetric PCR produces amplification products in which only one single strand contains fluorescent labels. Such asymmetric amplification products may be sequenced in double-stranded form. Symmetric PCR produces nucleic acid fragments in which both strands are fluorescently labeled. These two fluorescently labeled strands may be separated and immobilized separately in single-stranded form on support particles so that it is possible to determine the sequence of one or both complementary strands separately. As an alternative, any of the two strands may be modified on the 3′ end in such a way, for example by incorporating a PNA link, that removing monomeric building blocks by cleavage is no longer possible. In this case, double-strand sequencing is possible.

It is possible, where appropriate, to attach also a “sequence identifier”, i.e. a labeled nucleic acid of known sequence, to the nucleic acid strand to be studied, for example via enzymic reaction using ligase or/and terminal transferase, so that at the start of sequencing initially a known fluorescence pattern is obtained and only thereafter the fluorescence pattern corresponding to the unknown sequence to be studied is obtained.

The nucleic acid template whose sequence is to be determined may be selected, for example, from DNA templates such as genomic DNA fragments, cDNA molecules, plasmids, etc., or else from RNA templates such as mRNA molecules.

The fluorescent labeling groups may be selected from known fluorescent labeling groups used for labeling biopolymers, for example nucleic acids, such as, for example, fluorescein, rhodamine, phycoerythrin, Cy3, Cy5 or derivatives therefrom, etc.

Step (b) comprises introducing a loaded support particle into a sequencing device containing a microchannel. The support particle can be arrested in a capillary or a microchannel with the aid of a capturing laser, for example an IR laser, according to method step Cc). Methods of this kind are described, for example, in Ashkin et al. (Nature 330 (1987), 24-31) and Chu (Science 253 (1991), 861-866).

Preferably, the support particle is arrested using an automated process. For this purpose, the support particles are passed through the microchannel in a hydrodynamic flow, passing a detection element in the process. The detector in the detection window is adjusted so as to recognize a labeled sphere owing to the fluorescently labeled DNA located thereon and/or an additional fluorescently labeled probe and, as a result, to activate automatically the capturing laser in the measuring space. Support particles which have not been classified as positive by the detector can pass through. After capturing a support particle, the sorting process is stopped and the remaining support particles are removed by washing. This is followed by carrying out the sequencing reaction on the immobilized support particle.

The sequencing reaction of the method of the invention comprises progressively removing by cleavage individual nucleotide building blocks from the immobilized nucleic acid molecules. An enzymic cleavage is preferably carried out using an exonuclease, and it is possible to use single-strand and double-strand exonucleases degrading either in the 5′→3′ direction or in the 3′→5′ direction, depending on the type of immobilization of the nucleic acid strands on the support. Exonucleases which are particularly preferably used are T7 DNA polymerase, E. coli exonuclease I and E. coli exonuclease III.

The invention is based on passing the nucleotide building blocks released by the cleavage reaction through a microchannel by means of a hydrodynamic flow and determining them during their flow through said microchannel. The hydrodynamic flow makes it possible to increase the flow rate, and this in turn increases the probability of detection of a nucleotide building block. Furthermore, the hydrodynamic flow which is generated, for example, by suction action or by applying pressure can reduce the occurrence of wall effects, compared to electroosmotic pumping known in the prior art. The hydrodynamic flow through the microchannel preferably has a parabolic flow profile, i.e. the flow rate is highest in the center of the microchannel and then decreases with a parabolic function toward the edges down to a minimum rate. The flow rate through the microchannel, at maximum, is preferably in the range from 1 to 50 mm/s, particularly preferably in the range from 5 to 10 mm/s. The microchannel diameter is preferably in the range from 1 to 100 μm, particularly preferably from 10 to 50 μm. Preference is given to carrying out the measurement in a linear microchannel whose diameter is essentially constant.

Fluorescently labeled nucleotide building blocks can be identified according to step (e) of the method of the invention by means of any method of measurement, for example using space- or/and time-resolved fluorescence spectroscopy, which is capable of recording fluorescence signals down to single-photon counting in a very small volume element, such as one given in a microchannel.

It is possible, for example, to carry out detection by means of confocal single molecule detection, for example by fluorescence correlation spectroscopy, wherein a very small, preferably confocal, volume element, for example 0.1×10⁻¹⁵ to 20×10⁻¹² l of the sample fluid flowing through the microchannel is subjected to excitation light from a laser, which causes the receptors contained in said measuring volume to emit fluorescence light, and the fluorescence light emitted from the measuring volume is measured by means of a photodetector, followed by correlating the time-dependent change in the emission measured and the relative flow rate of the molecules involved so that it is possible, at an appropriately high dilution, to identify individual molecules in said measuring volume. For details of carrying out the method and of the devices used for detection, reference is made to the disclosure of European patent 0 679 251. Confocal single molecule determination is also described in Rigler and Mets (Soc.Photo-Opt.Instrum.Eng. 1921 (1993), 239 ff.) and Mets and Rigler (J. Fluoresc, 4 (1994) 259-264).

Alternatively or additionally, detection may also be carried out by time-resolved decay measurement, so-called time gating, as described, for example, by Rigler et al., “Picosecond Single Photon Fluorescence Spectroscopy of Nucleic Acids”, in: “Ultrafast Phenomenenes”, D. H. Auston, Ed., Springer 1984. Here the fluorescence molecules are excited in a measuring volume, followed by, preferably after an interval of ≧100 ps, opening a detection interval on the photodetector. In this way, it is possible to keep background signals generated by Raman effects sufficiently low in order to enable essentially interference-free detection.

In a particularly preferred embodiment, detection is carried out using a laser device which has a deflecting element or a phase-modulating element in the laser beam path, which, where appropriate in combination with one or more optical imaging elements, has been fitted for the purpose of generating from the laser beam a deflection pattern in the form of a linear or two-dimensional array of focal regions in the microchannel, the optical arrangement being fitted for the purpose, of projecting confocally each focal region for fluorescence detection by the photodetector arrangement. In a further preferred embodiment, the detection device is integrated into two walls facing one another and forming the microchannel, with one wall having an array of laser elements emitting into said microchannel as fluorescence excitation light source and the other one having an array of photodetector elements, in each case assigned to the opposite laser elements, as fluorescence light detectors. DE 100 23 423.2 discloses these two embodiments in detail.

An increase in the probability of detection of nucleotide building blocks, substantial to the invention, and thus an improvement in sensitivity is achieved by the hydrodynamic flow profile in the microchannel of the sequencing device. The hydrodynamic flow in the microchannel can be adjusted and regulated via suitable electronic controlling equipment. In addition to the hydrodynamic flow in the microchannel, electrophoretic and electroosmotic methods for transporting reagents may also be employed in the sequencing device. The method of the invention also allows parallel sequencing of a plurality of support particle-bound nucleic acid molecules in in each case different microchannels which are preferably arranged in parallel.

In a particularly preferred embodiment, the nucleic acid coupled to a support particle is essentially arrested in the center of the microchannel, the fluorescently labeled nucleotides removed by cleavage are directed in a laminar flow to a detection volume element downstream which is essentially positioned in the channel center which is the place with the highest flow rate. Preferably, the flow rate here is so high that the nucleotide arrives and is registered in the detector field, despite thermal broadening of the flow trajectories by Brownian diffusion. In this connection, the detector field is kept as small as possible so as to detect the nucleotide bases completely, while there is only a smallest possible fraction of background contamination (detector cross section to channel cross section ratio) in the detector.

The invention further relates to a device for sequencing an analyte in a sample fluid, comprising:
(a) an optically transparent microchannel,
(b) means for introducing a support particle on which a nucleic acid molecule has been immobilized into said microchannel, with essentially all nucleotide building blocks of at least one base type in at least one strand of said nucleic acid molecule carrying a fluorescent label,
(c) means for arresting said support particle at a predetermined position in said microchannel,
(d) means for generating a hydrodynamic flow in said microchannel,
(e) means for progressively removing by cleavage individual nucleotide building blocks from the immobilized nucleic acid molecule, and
(f) means for sequentially detecting the removed nucleotide building blocks.

The device furthermore preferably comprises automatic manipulation devices, heating or cooling equipment such as Peltier elements, means for sorting support particles, reservoirs and, where appropriate, supply lines for sample fluid and reagents and also electronic apparatuses for analysis.

The device is particularly suitable for carrying out the method of the invention.

Still a further embodiment of the invention relates to a method for single molecule sequencing using only two fluorescent labels. Thus the invention relates to a method for sequencing nucleic acids, which comprises the following steps:
(a) providing a support particle on which a nucleic acid molecule has been immobilized, with essentially all nucleotide building blocks in at least one strand of said nucleic acid molecule carrying a fluorescent label and with 2 fluorescent labels with different properties being used for the 4 bases,
(b) introducing said support particle into a sequencing device comprising a microchannel,
(c) arresting said support particle in said sequencing device,
(d) progressively removing by cleavage individual nucleotide building blocks from the immobilized nucleic acid molecule,
(e) passing the removed nucleotide building blocks through a microchannel and
(f) determining the base sequence of said nucleic acid molecule based on the sequence of said removed nucleotide building blocks.

In a preferred embodiment, at least two support particles having nucleic acid molecules immobilized thereon which have at least partially overlapping sequences and in which the two fluorescent labels used for the two-color sequencing are in each case assigned to different bases or/and base combinations. In a first variant, it is possible to assign on a support particle in each case a first fluorescent label to two bases B₁ and B₂ and a second fluorescent label to two bases B₃ and B₄, on a second support particle to assign in each case a first fluorescent label to two bases B, and B₃ and a second fluorescent label to two bases B₂ and B₄, and, where appropriate, on a third support particle to assign in each case a first fluorescent label to two bases B₁ and B₄ and a second fluorescent label to two bases B₂ and B₃. B₁, B₂, B₃ and B₄ represent the four different bases present in the nucleic acid to be sequenced, i.e. usually A, G, C and T.

In a further variant, on a first support particle in each case a first fluorescent label is assigned to a base B₁ and a second fluorescent label to three bases B₂, B₃ and B₄, on a second support particle in each case a first fluorescent label is assigned to a base B₂ and a second fluorescent label to three bases B₁, B₃ and B₄, on a third support particle in each case a first fluorescent label is assigned to a base B₃ and a second fluorescent label to three bases B₁, B₂ and B₄, and on a fourth support particle in each case a first fluorescent label is assigned to a base B₄ and a second fluorescent label to three bases B₁, B₂ and B₃.

In yet another variant, on a first support particle a first fluorescent label is assigned to two bases and a second fluorescent label to the other two bases, and on a second support particle a first fluorescent label is assigned to one base and a second fluorescent label to the other three bases. In this embodiment, it is possible, where appropriate, to use still further support particles having different 2/2 or/and 1/3 combinations.

The base sequence of a DNA sequence may also be completely determined by using only 2 fluorescent labeling groups with different spectroscopic properties such as, for example, emission wavelength or/and lifetime of the excited state. For this purpose, for example, two nucleotide bases can be provided with a first labeling group and the other two nucleotide bases with a second labeling group. Parallelly, the nucleic acid to be sequenced is labelled, in a parallel reaction, in such a way that other base combinations are provided with in each case the same labeling group. An example of this embodiment is listed below:

| | | |
|---|---|---|
| (1) | AT green, CG red: | ACGACATGCAATTGGGCAAAT |
| | | TGCTGTACGTTAACCCGTTTA |
| (2) | AC green, TG red: | ACGACATGCAATTGGGCAAAT |
| | | CATCACGTACCGGTTTACCCG |
| (3) | AG green, TC red: | ACGACATGCAATTGGGCAAAT |
| | | GTAGTGCATGGCCAAATGGGC |

A sequence can be obtained if AT, AC or AG are present in one color and CG, GT or CT are present in a different color (see combinations 1, 2 and 3). In order to obtain the complete base sequence, it is sufficient to sequence the color combinations 1 and 2, 2 and 3 or 1 and 3. A combination of 1, 2 and 3 is not absolutely needed. However, such a combination of 3 mixtures is sometimes convenient in order to reduce the probability of errors.

In the case of sequencing with two colors, there exist 2²×2²=16 possibilities for each color combination. As discussed above, it is possible to determine a complete sequence by using two dual base/fluorescent label combinations. However, for particular types of sequences, for example for the determination of mutations, a single dual combination may be sufficient.

Furthermore, it is possible, where appropriate, to use only a single base of the first color and the other three bases in a different color. In this case, four mixtures must be sequenced in order to obtain the complete sequence. A combination of the dual color combinations (e.g. two green and two red bases) with a single color combination (e.g. one green base and 3 red bases) is also interesting for particular embodiments.

The invention furthermore relates to a method for sorting particles in a microchannel, which comprises the following steps:
(a) passing particles through a detection element in said microchannel, said detection element being adjusted in such a way that a capturing laser, for example an IR laser, is activated if a predetermined parameter, for example a fluorescent label, is present on the particle and that said capturing laser is not activated if said predetermined parameter is not present on said particle,
(b) arresting a particle on which said predetermined parameter is present by said capturing laser in a measuring element,
(c) interrupting the sorting process and
(d) measuring the arrested particle.

The detection or/and measuring element are preferably confocal volume elements, the detection element being arranged in the microchannel upstream with respect to the measuring element. Measuring the arrested particle may comprise, for example, a sequencing as described above.

Furthermore, the invention is to be illustrated by the following figures in which:

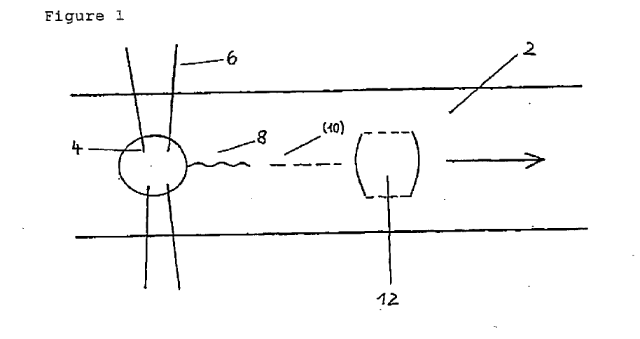

FIG. 1 depicts a section of a device for carrying out the method of the invention. In a microchannel (2) a support particle (4) is arrested by means of a capturing laser (6). On the support particle (4) a nucleic acid molecule (8) is immobilized from which individual nucleotide building blocks (10) are sequentially removed by enzymic digest and are transported by a hydrodynamic flow through the microchannel to a detection element (12), preferably a confocal detection element, and are detected there. The fluid flowing through the microchannel contains the enzyme used for digesting the immobilized nucleic acid molecule, preferably an exonuclease.

The flow rate through the microchannel is adjusted such that Brownian motion-caused broadening of the migration path of the nucleotide building blocks removed by cleavage is so low that said building blocks can be detected with sufficient statistical probability in the detection volume (12).

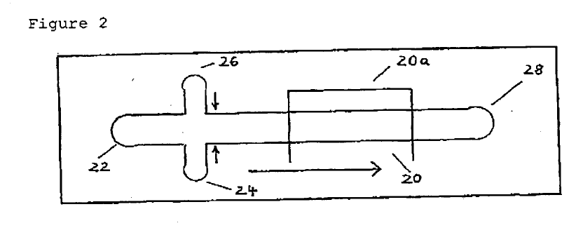

FIG. 2 depicts a larger section of the device of the invention, comprising the section (20a) of the microchannel (20), depicted in FIG. 1, with the capturing laser and the confocal detection element (not shown here). The device furthermore contains an inlet (22) and an outlet (28) for liquids, for example solvent, between which the hydrodynamic flow in the microchannel (20) is generated by applying pressure or by suction action. Furthermore, the device contains an opening for supplying support particles (24) and an opening for supplying enzyme (26). Enzyme and support particles may, where appropriate, be introduced by electroosmotic flow, with a negative electrode being applied at (24) and (26) and a positive electrode being applied at (28). Hydrodynamic flow through the microchannel (20) can be carried out by electronically controlled pumps which may be located outside the microstructure but may also be integrated therein.

The method is carried out by passing support particles through the opening (24) into the channel (20). A single support particle which is loaded with a nucleic acid molecule is arrested by the capturing laser. Other particles and contaminations are removed by washing. This is followed by adding enzyme through the opening (26) and carrying out the sequencing reaction. After sequencing has finished, the support particle is washed out of the microchannel. Thereafter, another sequencing cycle using a new microparticle can be carried out. This procedure may be automated using appropriate electronic controllers. Furthermore, the device may contain a plurality of microchannels for parallel sequencing of a plurality of support particles.

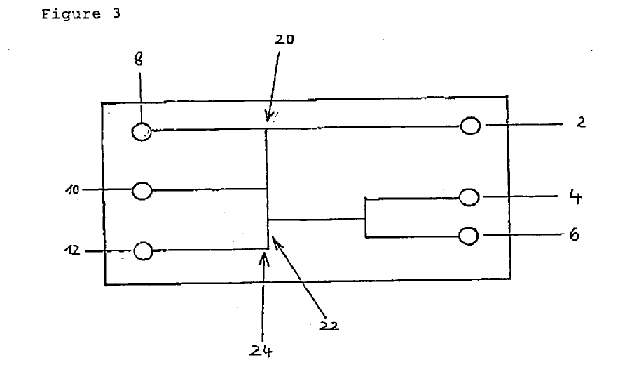

FIG. 3 depicts a preferred embodiment of an inventive device for single molecule sequencing. This device comprises a support with at least 6 openings for microfluidic channels. The opening (2) serves to supply the sample or the microparticles contained therein and, where appropriate, a buffer. The opening (4) serves to supply exonuclease. The opening (6) is provided for discharging used solution from the support. The openings (8) and (12) are likewise provided for discharging used solutions from the support. The opening (10) serves to supply buffer. Where appropriate, the device may contain still further openings for supplying or discharging liquids. The diameter of the microfluidic channels in the support is, in the case of the channels provided for supplying, preferably in the range from 40-80 μm, in particular approx. 50 μm. The discharging channels may have a considerably larger diameter, for example of up to 500 μm. The diameter may become wider immediately after the point of intersection of the channels and is intended to improve the control and to increase stability within the system.

The microparticle introduced into the device through the opening (2) is first captured in the region of position (20), for example by an IR laser, while the transport fluid is discharged from the support through opening (8). The captured microparticle is then transported within the support to position 22, for example by a liquid stream or/and by an IR laser, where it is again arrested, for example by an IR laser, and then subjected to enzymic degradation. For this purpose, enzyme is passed through the opening (4) to position (22) with the arrested microparticle and then transported out of the device again through the opening (12). The nucleotides released at position (22) by enzymic degradation of the nucleic acid on the microparticle are detected downstream at position (24).

After finishing the measurement, buffer may be introduced into the device through the opening (10) and discharged again through the openings (8) or/and (12). In this way, the enzyme present in the device is washed out through opening (12), preventing, at the same time, a new microparticle introduced into the device, for example at position (20) from contacting the enzyme prematurely.

Where appropriate, an electric field may be applied in the channels, for example via a positive electrode in the region of position (24) and a negative electrode in the region of opening (2). In this way it is possible to stretch the DNA to be sequenced which is immobilized on the microparticle and thus make it more accessible to enzymic treatment.

Reservoirs (not shown) are provided for the liquids to be introduced into the device and for the liquids discharged from the device.

Thus the invention further relates to a device for sequencing nucleic acids, comprising:
(a) an optically transparent microchannel,
(b) means for introducing a support particle on which a nucleic acid molecule has been immobilized into said microchannel, with essentially all nucleotide building blocks of at least one base type in at least one strand of said nucleic acid molecule carrying a fluorescent label,
(c) means for arresting said support particle at a first predetermined position in said microchannel,
(d) means for transporting said support particle to a second predetermined position of said microchannel,
(e) means for generating a flow in said microchannel,
(f) means for progressively removing by cleavage individual nucleotide building blocks from the immobilized nucleic acid molecule at the second predetermined position, and
(g) means for sequentially detecting the removed nucleotide building blocks.

In a preferred embodiment, this device comprises;
(a) a support comprising a system of microchannels which are in fluid communication with one another,
(b) an opening (2) for introducing a support particle having a nucleic acid molecule immobilized thereon into a microchannel, with essentially all nucleotide building blocks of at least one base type in at least one strand of said nucleic acid molecule carrying a fluorescent label,
(c) an opening (4) for feeding a nucleic acid-degrading enzyme into a microchannel,
(d) a plurality of openings (6, 8, 12) for discharging fluid from said support,
(e) an opening (10) for feeding buffer into a microchannel,
(f) means for generating a liquid stream in said microchannels,
(g) means for capturing the support particle at a first predetermined position (22),
(h) means for transporting a captured support particle to a second predetermined position (24),
(i) means for progressively removing by cleavage individual nucleotide building blocks from the immobilized nucleic acid molecule at the second predetermined position (24), and
(j) means for sequentially detecting the removed nucleotide building blocks.

An essential feature of this device is the fact that the support has means for transporting the particle containing the nucleic acid to be sequenced from a first predetermined position, the capturing position, to a second predetermined position, the degradation position. In this way, sequential operation of the device, i.e. successive analysis of a plurality of particles is facilitated, in particular because a contact with the nucleic acid-degrading enzyme can be prevented more easily in the capturing position.

In the device of the invention, microchannels in the direction of the discharge opening preferably have a diameter which is larger, preferably at least 1.5 times larger, than the diameter of microchannels in the direction of the supply openings. The flow within the device is preferably a hydrodynamic flow. Preference is furthermore given to providing means for supplying an electric field between the second predetermined position and the first predetermined position.

Finally, the invention relates to a method for sequencing nucleic acids, using a device as described above. This method preferably comprises the following steps:
(a) providing a support particle on which a nucleic acid molecule has been immobilized, with essentially all nucleotide building blocks of at least one base type in at least one strand of said nucleic acid molecule carrying a fluorescent label,
(b) introducing said support particle into an opening (2) of a support comprising a system of microchannels which are in fluid communication with one another,
(c) arresting said support particle at a first predetermined position (22) within said support,
(d) transporting said support particle to a second predetermined position (24) within said support,
(e) progressively removing by cleavage individual nucleotide building blocks from the immobilized nucleic acid molecule at said second predetermined position,
(f) passing the removed nucleotide building blocks through a microchannel, and
(g) determining the base sequence of said nucleic acid molecule based on the sequence of said removed nucleotide building blocks.

1-36. (canceled)

37. A method for sequencing nucleic acids, comprising:
(a) providing a support particle on which a nucleic acid molecule has been immobilized, with essentially all nucleotide building blocks of at least one base type in at least one strand of said nucleic acid molecule carrying a fluorescent label,
(b) introducing said support particle into a sequencing device comprising a micro channel,
(c) arresting said support particle in said sequencing device,
(d) progressively removing by cleavage individual nucleotide building blocks from the immobilized nucleic acid molecule,
(e) passing the removed nucleotide building blocks through a microchannel by means of a hydrodynamic flow and
(f) determining the base sequence of said nucleic acid molecule based on the sequence of said removed nucleotide building blocks.

38. The method as claimed in claim 37, wherein said support particle is made of a material selected form the group consisting of plastic, glass, quartz, metal, semimetal, metal oxides and a composite material.

39. The method as claimed in claim 37, wherein the diameter of the support particle is from 0.5 to 10 μm.

40. The method as claimed in claim 37, wherein the nucleic acid molecule is immobilized on the support particle via it 5′-terminus by means of bioaffinity interactions.

41. The method as claimed in claim 40, wherein a 5′-biotinylated nucleic acid molecule is immobilized to an avidine- or streptavidine-coated support particle.

42. The method as claimed in claim 37, wherein the nucleic acid molecule is immobilized in single-stranded form on the support particle.

43. The method as claimed in claim 37, wherein the nucleic acid molecule molecule is immobilized in double-stranded form on the support particle, it being possible for labeled nucleotide building blocks to be removed by cleavage only from one single strand.

44. The method as claimed in claim 37, wherein essentially all nucleotide building blocks of at least two base types carry a fluorescent label.

45. The method as claimed in claim 37, wherein the support particle is arrested using a capturing laser.

46. The method as claimed in claim 37, wherein the support particles are arrested in a microchannel.

47. The method as claimed in claim 37, wherein individual nucleotide building blocks are removed by cleavage by an exonuclease.

48. The method as claimed in claim 47, wherein T7 DNA polymerase, E. coli exonuclease I or E. coli exonuclease III is used.

49. The method as claimed in claim 37, wherein the removed nucleotide building blocks are passed through a microchannel having a diameter of from 1 to 100 μm.

50. The method as claimed in claim 37, wherein the removed nucleotide building blocks are passed through a microchannel with a velocity of from 1 to 50 mm/s.

51. The method as claimed in claim 37, wherein the determination is carried out by means of confocal fluorescence measurement in a detection volume element.

52. The method as claimed in claim 51, wherein the determination is carried out by means of confocal single molecule detection, such as, for example, fluorescence correlation spectroscopy.

53. The method as claimed in claim 37, wherein the determination is carried out by means of a time-resolved decay measurement or time gating in a detection volume element.

54. The method as claimed in claim 37, wherein nucleic acid molecules are determined in parallel in a plurality of microchannels.

55. The method as claimed in claim 37, wherein a sequence with known base sequence is attached to the nucleic acid molecule to be sequenced.

56. The method as claimed in claim 37, wherein the support particles are arrested essentially in the center of the microchannel and the removed nucleotide building blocks are directed in a laminar flow to a detection volume element which has been positioned in the center of the channel.

57. The method as claimed in claim 56, wherein the detection volume element is kept as small as possible in order to only just detect all removed nucleotide building blocks.

58. A device for sequencing an analyte in a sample fluid, which comprises:
(a) an optically transparent microchannel,
(b) means for introducing a support particle on which a nucleic acid molecule has been immobilized into said microchannel, with essentially all nucleotide building blocks of at least one base type in at least one strand of said nucleic acid molecule carrying a fluorescent label,
(c) means for arresting said support particle at a predetermined position in said microchannel,
(d) means for generating a hydrodynamic flow in said microchannel,
(e) means for progressively removing cleavage individual nucleotide building blocks from the immobilized nucleic acid molecule, and
(f) means for sequentially detecting the removed nucleotide building blocks.

59. A method for sequencing nucleic acids, which comprises the following steps:
(a) providing a support particle on which a nucleic acid molecule ahs been immobilized, with essentially all nucleotide building blocks in at least one strand of said nucleic acid molecule carrying a fluorescent label and with 2 fluorescent labels with different properties being used for the 4 bases.
(b) introducing said support particle into a sequencing device comprising a microchannel,
(c) arresting said support particle in said sequencing device.
(d) progressively removing by cleavage individual nucleotide building blocks from the immobilized nucleic acid molecule,
(e) passing the removed nucleotide building blocks through a microchanel and
(f) determining the base sequence of said nucleic acid molecule based on the sequence of said removed nucleotide building blocks.

60. The method as claimed in claim 59, wherein the different spectroscopic properties are selected from emission wavelength or/and lifetime.

61. The method as claimed in claim 59, wherein step (a) comprises providing at least 2 support particles on which nucleic acid molecules have been immobilized which have at least partially overlapping sequences and in which the 2 fluorescent labels, in each case, have been assigned to different bases or/and base combinations.

62. The method as claimed in claim 61, wherein a first support particle in each case a first fluorescent label is assigned to 2 bases, B₁ and B₂, and a second fluorescent label is assigned to B₃ and B₄, on a second support particle in each case a first fluorescent labile is assigned to 2 bases, B₁ and B₃, and a second fluorescent label is assigned to 2 bases, B₂ and B₄, and, where appropriate, on a third support particle in each case a first fluorescent label is assigned to 2 bases, B₁ and B₄, and a second fluorescent label is assigned to 2 bases, B₂ and B₃.

63. The method as claimed in claim 61, wherein on a first support particle in each case a first fluorescent label is assigned to a base B₁ and a second fluorescent label is assigned to 3 bases, B₂, B₃ and B₄, on a second support particle in each case a first fluorescent label is assigned to a base B₂ and a second fluorescent label is assigned to 3 bases, B₁, B₃ and B₄, on a third support particle in each case a first fluorescent label is assigned to a base B₃ and a second fluorescent label is assigned to 3 bases, B₁, B₂ and B₄, and on a fourth support particle in each case a first fluorescent label is assigned to a base B₄ and a second fluorescent label is assigned to 3 bases, B₁, B₂ and B₃.

64. The method as claimed in claim 61, wherein a first support particle a first fluorescent label is assigned to 2 bases and a second fluorescent label is assigned to the other 2 bases, and on a second support particle a first fluorescent label is assigned to one base and a second fluorescent label is assigned to the other three bases.

65. A method for sorting particles in a microchannel, which comprises the following steps:
(a) passing particles through a detection element in said microchannel, said detection element being adjusted in such a way that a capturing laser is activated if a predetermined parameter is present on the particle and that said capturing laser is not activated if said predetermined parameter is not present on said particle,
(b) arresting a particle on which said predetermined parameter is present by said capturing laser in a measuring element,
(c) interrupting the sorting process and
(d) measuring the arrested particle.

66. A device for sequencing nucleic acids, comprising:
(a) an optically transparent microchannel,
(b) means for introducing a support particle on which a nucleic acid molecule has been immobilized into said microchannel, with essentially all nucleotide building blocks of at least one base type in at least one strand of said nucleic acid molecule carrying a fluorescent label,
(c) means for arresting said support particle at a first predetermined position in said microchannel,
(d) means for transporting said support particle to a second predetermined position of said mircochannel,
(e) means for generating a flow in said microchannel,
(f) means for progressively removing by cleavage individual nucleotide building blocks from the immobilized nucleic acid molecule at the second position, and
(g) means for sequentially detecting the removed nucleotide building blocks.

67. A device for sequencing nucleic acids, comprising:
(a) a support comprising a system of microchannels which are in fluid communication with one another,
(b) an opening for introducing a support particle having a nucleic acid molecule immobilized thereon into a microchannel, with essentially all nucleotide building blocks of at least one base type in at least one strand of said nucleic acid molecule carrying a fluorescent label,
(c) an opening for feeding a nucleic acid-degrading enzyme into a microchannel,
(d) a plurality of openings for discharging fluid from said support,
(e) an opening for feeding buffer into a microchannel,
(f) means for generating a liquid stream in said microchannels,
(g) means for capturing the support particle at a first predetermined position,
(h) means for transporting a captured support particle to a second predetermined position,
(i) means for progressively removing by cleavage individual nucleotide building blocks from the immobilized nucleic acid molecule at the second predetermined position, and
(j) means for sequentially detecting the removed nucleotide building blocks.

68. The device as claimed in claim 67, wherein the diameter of the microchannels leading to the discharge openings is larger, preferably at least 1.5 times larger, than the diameter of the microchannels leading to the feeding openings and:

69. The device as claimed in claim 66, wherein the means (f) for generating a hydrodynamic flow are provided in the support.

70. The device as claimed in claim 66, wherein means for applying an electric field between the second predetermined position and the first predetermined position are provided.

71. A method for sequencing nucleic acids, characterized in that a device as claimed in claim 66, is used.

72. A method for sequencing nucleic acids, comprising:
(a) providing a support particle on which a nucleic acid molecule has been immobilized, with essentially all nucleotide building blocks of at least one base type in at least one strand of said nucleic acid molecule carrying a fluorescent label,
(b) introducing said support particle into an opening of a support comprising a system of microchannels which are in fluid communication with one another,
(c) arresting said support particle at a first predetermined position within said support,
(d) transporting said support particle to a second predetermined position within said support,
(e) progressively removing by cleavage individual nucleotide building blocks from the immobilized nucleic acid molecule at said second predetermined position,
(f) passing the removed nucleotide building blocks through a microchannel, and
(g) determining the base sequence of said nucleic acid molecule based on the sequence of said removed nucleotide building blocks.

73. The method as claimed in claim 38, wherein the diameter of the support particle is from 0.5 to 10 μm.

74. The method as claimed in claim 60, wherein step (a) comprises providing at least 2 support particles on which nucleic acid molecules have been immobilized which have at least partially overlapping sequences and in which the 2 fluorescent labels, in each case, have been assigned to different bases or/and base combinations.


SEQUENCE LISTING (3 sequences)

SEQ ID NO: 1 - DNA, 42 bp, Artificial Sequence
  example of a labeled nucleic acid molecule which gives a signal when sequenced
acgacatgcaattgggcaaattgctgtacgttaacccgttta

SEQ ID NO: 2 - DNA, 42 bp, Artificial Sequence
  example of a labeled nucleic acid molecule which gives a signal when sequenced
acgacatgcaattgggcaaatcatcacgtaccggtttacccg

SEQ ID NO: 3 - DNA, 42 bp, Artificial Sequence
  example of a labeled nucleic acid molecule which gives a signal when sequenced
acgacatgcaattgggcaaatgtagtgcatggccaaatgggc